(19) United States
(12) Patent Application Publication
(10) Pub. No.: US 2014/0370572 A1
(43) Pub. Date: Dec. 18, 2014

(54) MEANS AND METHODS OF INCREASING VIABILITY OF ROD-SHAPED BACTERIA

(71) Applicant: GENERAL MILLS, INC., Minneapolis, MN (US)

(72) Inventors: Bernhard H. Van Lengerich, Plymouth, MN (US); Martin Senz, Berlin (DE); Ulf Stahl, Berlin (DE); Edeltraud Mast-Gerlach, Berlin (DE)

(21) Appl. No.: 14/356,294

(22) PCT Filed: Nov. 12, 2012
(86) PCT No.: PCT/EP2012/072385

§ 371 Date: May 5, 2014

(30) Foreign Application Priority Data
Nov. 10, 2011 (EP) 11008965.3

(51) Int. Cl. C12N 1/20

(52) U.S. Cl. CPC C12N 1/20; C12N 2501/998
USPC 435/252.1

(57) ABSTRACT

This invention relates to use of peptone for controlling the volume and/or the length-to-diameter ratio of cells in culture, wherein said cells are cells of rod-shaped probiotic bacteria or rod-shaped fermentation bacteria.

This invention relates to use of peptone for controlling the volume and/or the length-to-diameter ratio of cells in culture, wherein said cells are cells of rod-shaped probiotic bacteria or rod-shaped fermentation bacteria.

In this specification, a number of documents including patent applications and manufacturer's manuals are cited. The disclosure of these documents, while not considered relevant for the patentability of this invention, is herewith incorporated by reference in its entirety. More specifically, all reference documents are incorporated by reference to the same extent as if each individual document was specifically and individually indicated to be incorporated by reference.

Lactic acid bacteria (LAB) are industrial important microorganisms and have been used as starter cultures for the manufacture of milk products as e.g. cheese, yoghurt or kefir since a long time. In the last decades increasing amounts of LAB are applied as probiotic supplements in functional food and animal nutrition. Among the used LAB the genus Lactobacillus (Lb.) is of great importance. For starter cultures as well as probiotics, a major challenge for manufacturers is to maintain vitality and viability of the organisms. A high viability of probiotics is of great interest, since the declared amount of living microorganism at the end of shelf-life of the probiotic food or pharmaceuticals is a main quality criterion. Given the widely accepted definition for probiotics of the FAO/WHO (Guidelines for the evaluation of probiotics in food. Joint FAO/WHO Working Group. Report on Drafting Guidelines for the Evaluation of Probiotics in Food London, Ontario, Canada (2002)), that probiotics are “live microorganisms which when administered in adequate amounts confer a health benefit on the host”, manufacturers try to produce cultures which are as robust as possible to withstand the conditions during the different processing steps, the storage and the passage through the gastrointestinal tract after consumption.

The term pleomorphism comes from the Greek pleion=more, morphe=figure and refers in bacteriology to growth forms of cells. It can be defined as variation of size and/or shape of a bacterial cell.

This phenomenon is well examined in the field of pathogenicity of microorganisms in medical microbiology; see Justice et al. (2008) (Morphological plasticity as a bacterial survival strategy; Nat Rev Microbiol, 6, 162-168). For example, filamentation is known to favor the transient entry and exit of epithelial cells and thus enhance the infectivity of many pathogenic bacteria like in urophatogenic Escherichia coli; see Mulvey et al. (1998) (Induction and evasion of host defenses by type 1-piliated uropathogenic Escherichia coli. Science, 282, 1494-1497); and Justice et al. (2004) (Differentiation and developmental pathways of uropathogenic Escherichia coli in urinary tract pathogenesis; Proc Natl Acad Sci USA, 101, 1333-1338). Further, the resistance to phagocytosis is well studied in pleomorphic fungi (Rooney and Klein (2002); Linking fungal morphogenesis with virulence; Cell Microbiol, 4, 127-137) and bacteria (Chauhan et al. (2006); Mycobacterium tuberculosis cells growing in macrophages are filamentous and deficient in FtsZ rings. J Bacteriol, 188, 1856-1865).

Investigations from Jeener and Jeener (1952) (Cytochemical observations on Thermobacterium acidophilus R26 after inhibition of growth by desoxyribonucleosides or uracil; Arch Int Physiol, 60, 194-195) with Lactobacillus (Lb.) acidophilus R-26 revealed that a DNA concentration below 0.25 μg/ml in the medium resulted, in addition to growth inhibiting effects, in elongation of the cells. These morphological variations were reversible within 3 h after adding DNA or uracil to the medium. Further, because of its unique requirement for deoxyribosides, Lb. acidophilus R-26 was used as assay organism for deoxyribosides by Siedler et al. (1957) (Studies on improvements in the medium for Lactobacillus acidophilus in the assay for deoxyribonucleic acid; J Bacteriol, 73, 670-675). Reich and Soska (1973) (Thymineless death in Lactobacillus acidophilus R-26. II. Factors determining the rate of the reproductive inactivation; Folia Microbiol (Praha), 18, 361-367) describe cellular death of Lb. acidophilus R-26 caused by lack of thymine as well as deoxyribosides in the medium. For both effects a loss of reproductive activity was held responsible. Also for Lb. acidophilus R-26, Soska (1966) (Growth of Lactobacillus acidophilus in the absence of folic acid; J Bacteriol, 91, 1840-1847) demonstrated the termination of DNA synthesis after transferring the culture in a medium lacking thymine or deoxyribosides. Nevertheless cells grew in length and cell number increased only two to four times. Additionally, Soska (1996) found that a decrease of the phosphate concentration to one-fortieth resulted in cells which were only one-third to one-half as large. Beck et al. (1963) (Purification, kinetics, and repression control of bacterial trans-N-deoxyribosylase; J Biol Chem, 238, 702-709) demonstrated growth dependency on at least one exogenous deoxyribonucleoside for Lactobacillus strains Lb. leichmannii, Lb. lactis, Lb. acidophilus and Lb. delbrueckii. Limiting nutrition conditions were associated with the formation of filamentous cells and enhanced activities of trans-N-deoxyribosylase (EC 2.4.2.6), an enzyme which is involved in the DNA synthesis and found in bacteria that require external deoxyribonucleosides for growth. This enzyme was found only in the four pleomorphic strains mentioned above, compared to the two other investigated organisms Lb. casei and Escherichia coli_{15T}, which did not form filamentous variants. The results were confirmed from Sawula et al. (1974) for Lb. acidophilus R-26.

Deibel et al. (1956) (Filament formation by Lactobacillus leichmannii when desoxyribosides replace vitamin B12 in the growth medium; J Bacteriol, 71, 255-256) reported that cells of Lb. leichmannii 313, grown in a medium with a vitamin B₁₂ concentration of 0.02 ng/ml as well with a thymidine concentration of 0.5 mg/ml, had a filamentous like cell morphology. At concentrations of 0.5 ng/ml for vitamin B₁₂ and 5.0 mg/ml for thymidine, propagated cells formed normal rods. The authors discussed that both components likely play an important role in cell division. Similar results were found by Kusaka and Kitahara (1962) (Effect of several vitamins on the cell division and the growth of Lactobacillus delbrueckii; J Vitaminol (Kyoto), 8, 115-120) for Lb. delbrueckii No. 1. They observed cell elongation at a vitamin B₁₂ concentration of 0.3 ng/ml while a concentration as high as 1 μg/ml was required for cellular division. This discrepancy is considered to be the reason for abnormal cell elongation of Lb. delbrueckii. Dave and Shah (1998) (Ingredient supplementation effects on viability of probiotic bacteria in yogurt. J Dairy Sci, 81, 2804-2816) describe an investigation into the effects of L-cysteine, whey powder, whey protein concentrate, acid casein hydrolysate and tryptone on viability of probiotic bacteria in yogurt.

Webb (1949a) (The Influence of Magnesium on Cell Division: The Effect of Magnesium on the Growth and Cell Division of Various Bacterial Species in Complex Media; J Gen Microbiol, 3, 410-417) and Webb (1949b) (The influence of magnesium on cell division; the effect of magnesium on the growth of bacteria in simple chemically defined media; J Gen Microbiol, 3, 418-424) investigated the phenomenon of pleomorphism caused by magnesium deficiency for certain species of Clostridium and Bacillus. In these studies, inhibition of cell division caused by magnesium deficiency was presumed to induce filamentation of the normally rod-shaped bacteria. Wright and Klaenhammer (1981) (Calcium-Induced Alteration of Cellular Morphology Affecting the Resistance of Lactobacillus acidophilus to Freezing; Appl Environ Microbiol, 41, 807-815) demonstrated that calcium supplementation of the growth medium induced enhanced stability of Lb. acidophilus NCFM concentrates during freezing. The authors observed that calcium supplemented MRS broth (de Man et al., (1960); A Medium for the Cultivation of Lactobacilli; J. Appl. Bact. 23, 130-135) caused a morphological transition of the culture from filamentous to bacilloid rods, and related the calcium induced morphology change to a stability increase. Interestingly same authors demonstrated later (Wright and Klaenhammer (1983a); Survival of Lactobacillus bulgaricus During Freezing and Freeze-Drying After Growth in the Presence of Calcium. Journal of Food Science, 48, 773-777) that the addition of calcium in the growth medium of the two strains Lb. bulgaricus 1234-O and F also induced enhanced stabilities during freezing and freeze-drying, but in these studies calcium supplementation had no effect on cell morphology or growth. Manganese and magnesium salts failed to exert protective effects in these investigations. In further experiments, the same authors investigated the influence of phosphate concentration on growth, acid production and cellular morphology of the two strains Lb. bulgaricus 1243-F and 1489 (Wright and Klaenhammer (1984); Phosphated Milk Adversely Affects Growth, Cellular Morphology, and Fermentative Ability of Lactobacillus bulgaricus; J. Dairy Sci., 67, 44-51). In addition to an inhibition of acid production and growth, cellular morphology of both strains changed when cultured in milk containing 2 to 3% phosphate or commercial phage inhibitory medium. These media induced a transition to long chains of connected cells compared to normal short rods growing in non-supplemented milk. The observed poor growth and alteration of cellular morphology were related to the requirement for divalent cations for a proper growth and cell assembly and are in accordance with earlier results from the authors (Wright and Klaenhammer (1983b); Influence of Calcium and Manganese on Dechaining of Lactobacillus bulgaricus; Appl Environ Microbiol, 46, 785-792), where calcium and manganese were predicted to be necessary for an adequate dechaining activity of the corresponding enzymes. Similar results were observed by Kojima (1970a) (A study on the pleomorphism of Lactobacillus bifidus; Kobe Daigaku Igakubu Kiyo, 32, 126-147) and Kojima et al. (1970b) (Necessity of calcium ion for cell division in Lactobacillus bifidus; J Bacteriol, 104, 1010-1013), who emphasize an indispensable role of calcium ions for cell division in Lb. bifidus. The authors imaged calcium induced septum formation via electron microscopy.

Altermann et al. (2004) (Identification and phenotypic characterization of the cell-division protein CdpA; Gene, 342, 189-197) were able to knock out the open reading frame ORF_{—}223 in Lb. acidophilus NCFM, which encodes for the cell separating protein cdpA. These mutants, in which cell division was inhibited, generated long cell chains in which the single cells were about two to three times larger in terms of volume than wild type cells. Further, those cells are less stable against NaCl—, osmotic- and ethanol-stress than the wild type but were more stable against oxgall-treatments. Rechinger et al. (2000) (“Early” protein synthesis of Lactobacillus delbrueckii ssp. bulgaricus in milk revealed by [35S] methionine labeling and two-dimensional gel electrophoresis; Electrophoresis, 21, 2660-2669) observed for Lb. delbrueckii spp. bulgaricus that bacteria grown in complex media like MRS or reconstituted skim milk had normal rod shapes whereas the use of a chemical defined medium resulted in filamentous cells, indicating a lack of at least one factor which is present in the complex media. Suzuki et al. (1986) (Growth of Lactobacillus bulgaricus in Milk. 1. Cell Elongation and the Role of Formic Acid in Boiled Milk; J. Dairy Sci., 69, 311-320) demonstrated an abnormal elongation of Lb. bulgaricus B5b when grown in milk, which was boiled for 15 min at 100° C. This procedure reduced the nutrition content in the milk which was responsible for a repression of the bulk RNA synthesis and hence a defective cell division progress. Rhee and Pack (1980) (Effect of environmental pH on chain length of lactobacillus bulgaricus; J Bacteriol, 144, 865-868) reported chain-generation in Lb. bulgaricus NLS-4 at alkaline pH values (above 7.5) in a steady-state continuous culture. The authors could correlate this phenomenon to inhibition of the synthesis of the dechaining enzyme(s) at enhanced pH values.

While the prior art in several instances describes correlations between presence or absence of certain agents on the one hand and the occurrence of pleomorphic forms on the other hand, little is known about how cellular morphology may be controlled in a systematic manner with the aim of achieving superior biotechnological products such as probiotic products or fermentation starters. The technical problem underlying the present invention may therefore be seen in the provision of improved means and methods of culturing microorganisms.

This technical problem is solved by the subject-matter of the enclosed claims.

In a first aspect, this invention relates to use of peptone for controlling the volume and/or the length-to-diameter ratio of cells in culture, wherein said cells are cells of rod-shaped probiotic bacteria or rod-shaped fermentation bacteria.

The term “peptone” has the meaning as established in the art. It refers to a mixture of peptides and amino acids which may be obtained by degradation from animal or plant proteins as starting material. The degradation giving rise to peptides and amino acids may be effected by chemical hydrolysis, e.g. caused by acids and/or by enzymatic digestion, preferably with pepsin. Pepsin occurs in several isoforms, pepsin A, pepsin B and pepsin C being the prominent ones. The corresponding enzyme commission (EC) numbers are 3.4.23.1, 3.4.23.2 and 3.4.23.3. If not specified otherwise, “pepsin” refers to pepsin A. Commercially available pepsin is usually pepsin A obtained from porcine stomach. Alternatively, enzymatic digestion may be effected with trypsin or other endopeptidase(s). Peptones are typical constituents of media for microorganisms such as bacteria or yeasts.

Preferred peptones are described below.

The term “rod-shaped bacteria” is established in the art and refers to bacteria which share a common morphology. The term is not to be confused with a taxonomic criterion. The genus Bacillus is a characteristic representative of rod-shaped bacteria. As will be further detailed in the following, a rod can be described in geometrical terms as follows: an open cylinder with a half sphere at either end such that a closed convex compartment is formed. Sometimes the term “bacilli” is used to denote any rod-shaped bacteria in which case it is not to be confused with the taxon Bacillus. Rod-shaped bacteria are to be distinguished from spherical or ellipsoid bacteria. On the right hand side of FIG. 3, rods of various length can be seen. Longer rods are sometimes also referred to as filamentous forms, whereas short rods are sometimes also referred to as bacilloid forms.

The stable rod-shaped structure arises from the presence of a cell wall which is more rigid than the cell membrane. The cell wall is predominantly made of peptidoglycans which give rise to a structure which is more rigid than the lipid bilayer of the cell membrane. Between the cell wall on the outside and the cell membrane in the interior, there is a lumen also referred to as periplasmic space.

The size of a rod-shaped bacterium may be defined in terms of its volume. Instrumentation for determining cellular volumes is at the skilled person's disposal and described in the examples. The terms “volume” and “cellular volume” are used interchangeably.

Given the definition of a rod in geometrical terms as provided above, it is apparent that a single parameter may be used to define the overall shape of a given rod. This parameter is the length-to-diameter ratio abbreviated as “L/D ratio”. Means for determining the length-to-diameter ratio will be described in the following. In particular, in a first step the volume of the cells at issue is determined, and in a second step the L/D ratio is calculated therefrom.

In the course of calculating the L/D ratio, one option is to assume a particular cellular diameter. In case of the Lactobacillacaea, in particular in case of Lactobacillus acidophilus, but not confined thereto, a value of 1 μm is a good estimate of the cellular diameter D. In the case rod-shaped bacteria, it is understood that the diameter D refers to the diameter of the cylindrical part of the rod. The inventors furthermore observed that, depending on the culture medium used, the average cell volume varies. To a good approximation, the variation of cellular volume arises from a variation of rod length but not of rod diameter. The average cellular volume, as a function of radius r and length h of the cylindrical part of the rod is defined as follows: V=π r² h+4/3 π r³. Assuming, as stated above, that D is 1 μm and the radius r=0.5 μm, it follows that h can be determined from cellular volume V. Since the total length of the rod L=h+2 r (two half spheres at the ends of the cylindrical part of the rod), and that the diameter D=2 r=1 μm, it follows that the L/D ratio can be determined and used as a parameter characterizing the morphology of the rod-shaped bacteria according to the present invention.

In accordance with the invention, the rod-shaped bacteria are further defined to be either rod-shaped probiotic bacteria or rod-shaped fermentation bacteria. As mentioned herein above, probiotics are alive microorganisms which, when administered in adequate amounts, confer a health benefit on the host. Preferred taxa falling under said definition are detailed herein below. As is apparent from the definition of probiotics, it is important that probiotic bacteria, when delivered to the host and when they arrive at their destination are alive.

The term “rod-shaped fermentation bacteria” refers to any rod-shaped bacteria capable of fermentation. The term “fermentation” as used herein has the usual meaning as established in the art and refers to the biochemical process of oxidation of organic compounds, thereby extracting energy such as in the form of ATP. In the course of oxidation as part of fermentation processes, an endogenous electron acceptor is used. The latter aspect distinguishes fermentation from respiration. Preferably, said rod-shaped fermentation bacteria are rod-shaped bacteria as they are used in biotechnological production processes. Such biotechnological production processes include the production of beverages, food for human or animal consumption, dietary supplements, functional food and medicinal products. Preferred taxa falling under the above definitions are provided below.

The present invention as a whole is furthermore applicable to rod-shaped bacteria used as biocontrol agents, in particular as biopesticides and biopreservatives. The term “biocontrol agent” refers to microorganisms—as opposed to chemicals—which may be used for controlling other microorganisms. Bacillales are useful as biocontrol agents. Examples of biopesticides include Bacillus thuringiensis ssp. aizawai. Examples of biopreservatives include Lactobacillus plantarum.

A further group of target cells belonging to fermentation bacteria are cells of rod-shaped bacteria as comprised in fermentation starters, sometimes also referred to as “starter cultures”. As is known in the art, fermentation starters are preparations which assist the beginning of the fermentation process in preparation of various foods and fermented drinks. Bacteria and/or yeasts are comprised in typical fermentation starters. Preferred bacteria as comprised in said fermentation starters are defined in terms of taxa herein below.

The term “culture”, when used as part of the term “starter culture” has a special meaning in that it refers to a composition comprising one or more species of microorganism which are suitable to start fermentation. Otherwise, and generally speaking, the term “culture” has the meaning as established in the art and refers on the one hand to a method of multiplying microbial organisms by letting them reproduce in predetermined culture media under controlled laboratory and/or production conditions, and on the other hand to the composition of matter where the culture actually occurs, said composition of matter comprising culture medium and microorganisms. The term “culture” as used herein refers to culture on any scale, contained or held in any vessel or carrier, and any state of matter. For example, culture may be liquid culture. “Culture” may also extend to the presence, preferably the propagation of microorganisms in compositions obtained by fermentation, such compositions including beverages, food, dietary supplements and medicinal products. In a preferred embodiment, the term “culture” relates to the step of cultivating in a culture medium to which peptone has been added or which comprises peptone.

As noted above, peptones are typical constituents of culture media. The present inventors surprisingly discovered that the choice of peptone is a means of influencing cellular volume and a specific morphological parameter in specific microorganisms, the morphological parameter being the length-to-diameter ratio, and the microorganisms being the above mentioned specific rod-shaped bacteria. Said “influencing” is statistically significant and also referred to as “controlling” herein. Length of the rods and cellular volume have been found to depend significantly on the choice of the peptone comprised in the culture medium. By decreasing volume and/or L/D ratio, high cell counts or concentrations are achieved; see, for example, the data shown in FIG. 1. Moreover, as will be discussed further below, decreasing volume and/or length-to-diameter ratio are a means of rendering the rod-shaped bacteria as defined herein more viable and resistant to mechanical, chemical or thermal stress conditions as they may occur in biotechnological production processes.

In a preferred embodiment, said controlling is decreasing and said peptone is fat stock peptone, preferably peptone of porcine origin, more preferably a peptic digest of porcine origin. The term “fat stock peptone” refers to peptone originating from fat stock. Fat stock peptone may be obtained by hydrolyzing or digesting protein or protein-containing tissue, in particular meat of fat stock. The term “fat stock” refers to animals that are slaughtered such as pig and cattle (including Bos primigenius taurus). Peptone from milk including peptone from casein is not to be subsumed under “fat stock peptone”. The term “porcine origin” refers to any tissue obtained from fat stock of the genus Sus, preferably the species Sus scrofa, most preferably Sus scrofa domestica.

The present inventors surprisingly found that, by choosing a fat stock peptone, preferably peptone of porcine origin, cellular morphology of the rod-shaped bacteria according to the present invention can be modified such that bacterial cells of smaller volume and/or shorter rods (smaller L/D ratios) are obtained as well as higher cell concentrations. Preferably, said peptone is a peptic digest of porcine tissue. A peptic digest is a preparation obtained from starting material upon the addition of the enzyme pepsin. As regards said starting material, preference is given to tissues, in particular meat, of fat stock, in particular of porcine origin, more specifically to stomach tissue of porcine origin. At the same time, the use of other protein sources or protein comprising tissues of porcine origin is envisaged.

While peptones are primarily considered as sources of amino acids and peptides, it is at the same time true that they comprise other constituents since entire tissues are typically used in their preparation. As regards these other constituents, preference is given to peptones with a high concentration of nucleic acid building blocks such as nucleotides, nucleosides and nucleobases as well as their derivatives. As an indicator of such high concentrations, the concentration of thymidine and/or hypoxanthin may be used. Thymidine and hypoxanthin as such are preferably present in high concentrations as well. High concentrations of hypoxanthin are concentrations above 50, 100, 150 or 200 μg/g. High concentrations of thymidine are concentrations above 20, 40, 60 or 70 μg/g. It is envisaged to use peptones fulfilling any of these criteria (high concentrations of nucleic acid building blocks, thymidine and/or hypoxanthin), which peptones are not necessarily confined to fat stock peptone or peptone of porcine origin.

In the course of the present invention, the inventors in part prepared their own media by using peptones of different origin and/or from different manufacturers. A specific peptone of porcine origin which performed in a particularly outstanding manner is Bacto™ Proteose Peptone No. 3, available from Becton Dickinson, which previously has been known as Difco™ Proteose Peptone No. 3. Despite the name change, the method of manufacture of said peptone has not been changed. Further information on Bacto™ Proteose Peptone No. 3 can be found, for example, in the third Edition BD Bionutrients™ technical manual (October 2006); see in particular the Tables at pages 9, 42 and 43 thereof. Bacto™ Proteose Peptone No. 3 is a particularly preferred peptone of porcine origin for all aspects of this invention. Bacto™ Proteose Peptone No. 3 is sometimes briefly referred to as Proteose Peptone No. 3 or Peptone No. 3 herein.

In a further preferred embodiment, the average volume of said cells in the presence of said peptone is below 3 μm³, preferably between 2 and 3 μm³, more preferably between 1.4 and 2 μm³, and most preferred between 1.1 and 1.4 μm³, further preferred cell volumes including 1.0, 1.2, 1.3 and 1.5 μm³; and the average length-to-diameter ratio is below 5, preferably below 4 or below 3 or below 2.5, more preferably below 2.2 or below 2.1 or below 2.0, and most preferred below 1.8. FIG. 1 shows the average cellular volume (“mean cell volume”) as well as the cell count per ml for a variety of different culture conditions. FIG. 3 shows distributions of cellular volume as determined for culture in different media.

In a preferred embodiment, the average length-to-diameter ratio is at least 1.1, 1.2, 1.3, 1.4 or 1.5. In any case, a value above 1.0 is implied by the term “rod-shaped” which term requires a cylindric structure being present; see above.

In a second aspect, the present invention provides the use of fat stock peptone, preferably peptone of porcine origin, more preferably a peptic digest of porcine origin, for increasing viability, stability, shelf-life, DNA replication, septum formation and/or cell division of cells, wherein said cells are cells of rod-shaped probiotic bacteria or rod-shaped fermentation bacteria.

This embodiment relates to a further surprising finding of the present inventors, namely that specific means which allow to control the cellular volume or the length-to-diameter ratio, namely the peptone according to the invention, furthermore provide for increasing the quality of a culture of cells as well as of compositions or preparations comprising said cells or obtained therefrom. Quality parameters according to the present invention are viability, vitality, stability, shelf-life, DNA replication, septum formation and cell division.

The term “viability” of cells denotes their status to be alive. That status can be expressed by surviving, growing and multiplying of cells and is for many issues verifiable by a positive cultivability. The term “vitality” of cells denotes their status to have a designated metabolic activity. The term “stability” as used herein relates to the capability of maintaining viability over a certain time period or after processing, processing including extruding, lyophilizing, freezing, drying and storage.

“Shelf-life” is parameter frequently used to characterize commercially available products. In the present case, the term is used to designate the period of time until which a probiotic culture or a preparation obtained therefrom is capable of conferring the above mentioned health benefit on the host, or, to the extent reference is made to fermentation bacteria, to the capability of the latter to perform the desired fermentation process. The latter three quality parameters (DNA replication, septum formation and cell division) can be seen as microscopic or biochemical indicators of viability. The term “septum” refers to the boundary which is formed between dividing cells in the course of cell division. One or more of the above mentioned quality parameters may be improved when using the specific peptone according to the present invention.

In a further aspect, the present invention provides a method of selecting a cell culture medium which medium increases viability of cells or stability or shelf-life of a preparation comprising cells cultured in said medium, wherein said cells are cells of rod-shaped Gram-positive bacteria, preferably rod-shaped probiotic bacteria or rod-shaped fermentation bacteria, said method comprising determining the average volume and/or the average length-to-diameter ratio of said cells in culture, wherein low average volumes or low average length-to-diameter ratios are indicative of a suitable medium, preferred average volumes and average length-to-diameter ratios being as defined herein above.

The term “Gram-positive” is well-established in the art. It refers to the capability of certain bacteria, namely Gram-positive bacteria, to retain crystal violet staining upon decolorization with ethanol. This capability does not occur in Gram-negative bacteria. The capability of Gram-positive bacteria to retain the crystal violet stain is attributed to the presence of a thick cell wall rich in peptidoglycans. Bacifiales, Lactobacillales and Bifidobacteriales are all Gram-positive bacteria.

This aspect of the invention relates to a screening method which allows the identification of suitable cell culture media. While the previous aspects relate to rod-shaped probiotic bacteria or rod-shaped fermentation bacteria, and furthermore are confined to peptones as controlling agents, the method of selecting a cell culture medium according to the invention is not confined to a specific agent such as a peptone. As a consequence, it is applicable to rod-shaped Gram-positive bacteria in general. This particular aspect of the invention arises from the surprising finding that lowering the average cellular volume and/or the average length-to-diameter ratio in a culture of rod-shaped Gram-positive bacteria is a means of increasing viability, stability and/or shelf-life.

In a preferred embodiment, the source of amino acids and/or peptides in said medium is varied in the course of said method of selecting a cell culture medium. In particular, it is envisaged to compare enzymatic digests such as peptic digests of animal protein sources, in particular meat of animals including fat stock meat.

The present invention, in a further aspect, relates to a method of establishing an average volume and/or average length-to-diameter ratio of cells in culture, wherein said cells are cells of rod-shaped probiotic bacteria or rod-shaped fermentation bacteria, which average volume is below 3 μm³, preferably between 2 and 3 μm³, more preferably between 1.4 and 2 μm³, and most preferred between 1.1 and 1.4 μm³ and which average length-to-diameter ratio is below 5, preferably below 4 or below 3 or below 2.5, more preferably below 2.2 or below 2.1 or below 2.0, and most preferred below 1.8, and which method comprising culturing said cells in the presence of fat stock peptone, preferably peptone of porcine origin, more preferably a peptic digest of porcine origin.

In a further aspect, the present invention provides a method of culturing said cells in the presence of fat stock peptone, preferably peptone of porcine origin, more preferably a peptic digest of porcine origin for a certain time span. The cultivation time preferable proceeds till a maximal concentration of viable cells and a minimum of the averaged cell volume is reached. The preferred stage of the culture is the so called stationary growth phase, wherein that phase is reached between 10 and 48 h, preferable between 12 and 24 h, more preferable between 14 and 22 h, and most preferable between 16 and 20 h.

In a further aspect, the present invention provides a method of increasing viability, stability, shelf-life, DNA replication, septum formation and/or cell division of cells, wherein said cells are cells of rod-shaped probiotic bacteria or rod-shaped fermentation bacteria, wherein said method comprises culturing said cells in the presence of fat stock peptone, preferably peptone of porcine origin, more preferably a peptic digest of porcine origin.

In a preferred embodiment of the uses and methods disclosed above, said probiotic bacteria or fermentation bacteria are selected from rod-shaped Lactobacillales and rod-shaped Bifidobacteriales, preferably rod-shaped Lactobacillaceae and rod-shaped Bifidobacteriaceae, more preferably Lactobacillus and Bifidobacterium. Further preferred taxa are rod-shaped Bacillales, preferably rod-shaped Bacillaceae, a preferred genus being Bacillus; and rod-shaped Clostridiales, preferably Clostridium.

Preferably, said Lactobacillus is selected from the group consisting of Lactobacillus acidophilus, Lactobacillus casei, Lactobacillus delbrueckii, Lactobacillus johnsonii, Lactobacillus rhamnosus and Lactobacillus salivarius.

In a more preferred embodiment, (a) Lactobacillus acidophilus is Lactobacillus acidophilus or Lactobacillus acidophilus NCFM; (b) Lactobacillus casei is Lactobacillus casei subsp. rhamnosus (ATCC 7469); (c) Lactobacillus delbrueckii is Lactobacillus delbrueckii subsp. lactis or Lactobacillus delbrueckii subsp. bulgaricus; (d) Lactobacillus johnsonii is Lactobacillus johnsonii La1; (e) Lactobacillus rhamnosus is Lactobacillus rhamnosus GG (ATCC 53103); or (f) Lactobacillus salivarius is Lactobacillus salivarius subsp. salivarius.

Further preferred species and strains from the genus Lactobacillus (“L.”) and Bifidobacterium (“B.”) are L. acidophilus R0052 (Lallemand), L. casei shirota (Yakult), L. casei immunitas (DN114001) (Danone), L. paracasei CRL431 (Chr. Hansen), L. paracasei ST11 (Nestle), L. paracasei LP33 (GenMont Biotech), L. paracasei F19 (Medipharm), L. plantarum 299V (Probi AB/Lallemand), L. gasseri (Merck/Seven Seas), L. reuteri SD2112 (Biogaia), L. rhamnosus LGG (Valio), L. rhamnosus GR-1 (Urex Biotech), L. rhamnosus 271 (Probi AB), L. salivarius UCC118 (University of Cork), L. helveticus CPN4 (Calpis, Japan), L. helveticus (LKB16H) (Valio), Lactococcus lactis L1A (Essum AB), B. lactis (DN 173 010) (Danone), B. lactis Bb-12 (Chr. Hansen), B. longum BB-536 (Morinaga), B. longum Rosell 152 (Lallemand), B. longum SBT-2928 (Snow Brand Milk Prod., Japan), B. breve strain (Yakult), B. lactis HN019 (Howaru, Danisco).

Particularly preferred are Lactobacillus acidophilus NCFM, Lactobacillus acidophilus, Lactobacillus casei subsp. rhamnosus, Lactobacillus rhamnosus GG and Lactobacillus delbrueckii subsp. lactis.

In a further preferred embodiment, said peptone is comprised in culture medium such as MRS medium, preferably BD Difco™ Lactobacilli MRS broth, or GEM medium. MRS medium is known in the art and has been described in the publication by de Man et al. (1960) (de Man, J. D., Rogosa M. and Sharpe M. E. (1960) A Medium for the Cultivation of Lactobacilli. J. Appl. Bact. 23, 130-135). Various MRS media have been tested by the inventors which MRS media differ from each other as regards the comprised peptone. A preferred MRS medium, designated herein “MRSD” is an MRS medium comprising Proteose Peptone No. 3 which contributes to the particularly good performance of MRSD medium. The constituents of MRSD medium are provided in Example 1 herein below. In an alternative preferred embodiment, GEM (general edible medium) as described in Saarela et al. (2004) (Stationary-phase acid and heat treatments for improvement of the viability of probiotic lactobacilli and bifidobacteria; J Appl Microbiol, 96, 1205-1214) is used. Typically, GEM contains soy peptone; see Example 1. The other constituents of GEM are also provided in Example 1. According to the invention, soy peptone may be replaced with any other peptone, wherein preference is given to peptones of porcine origin, in particular Proteose Peptone No. 3.

Generally speaking, concentrations of said peptone in the range from 5 to 50, 10 to 40, 12 to 30, or 15 to 25 g/I are preferred.

In a more preferred embodiment, (a) said MRS medium comprises 5 to 20 g/l, preferably about 10 g/l of said peptone; or (b) said GEM medium comprises 10 to 50 g/l, preferably 20 to 40 g/l, more preferably about 30 g/l of said peptone. As stated above, a particularly preferred peptone is Bacto™ Proteose Peptone No. 3.

To the extent GEM medium is employed, it is preferred that said GEM medium furthermore comprises Tween 80, preferably in a concentration of 0.5 to 2 g/l, more preferably about 1 g/l.

In further preferred embodiments of the uses and methods of the present invention (a) viability is viability in culture or in a preparation; (b) stability is stability in a preparation; (c) shelf-life is shelf-life in a preparation; (d) DNA replication is DNA replication in culture; (e) septum formation is septum formation in culture; and (f) cell division is cell division in culture.

In a preferred embodiment, said preparation is selected from preparations wherein said cells are encapsulated or embedded in a protective matrix, such as extrudates or spheres; lyophilisates; frozen preparations; and dried preparations.

It is known in the art that viability of rod-shaped bacteria as defined herein above, in particular rod-shaped probiotic bacteria may be further increased by encapsulating or embedding them into a protective matrix. A preferred process of encapsulating or embedding is extruding. A preferred protective matrix is a dough. Further or alternative constituents of said protective matrix may be skimmed milk or LyoA; see also Example 2. “LyoA” is used herein to designate an aqueous solution of Gelatine (1.5% (w/w)), glycerol (1% (w/w)), Maltodextrin, preferably Glucidex12® (5% (w/w)) and lactose monohydrate (5% (w/w)).

The process of extrusion, giving rise to extrudates, is known in the art. Generally speaking, a gel or a viscous or dough-like composition is squeezed through an orifice. The manufacture of pasta is an example of extruding. According to the present invention, preference is given to extrusion under mild conditions, also referred to as “cold extrusion”. To the extent necessary, cooling is applied during the extrusion process, said cooling serving to keep the temperature in a range of about 20° C. to about 15° C. If rod-shaped bacteria according to the present invention are combined with a dough, and the dough is extruded, the bacterial cells will be immobilized within the network, in particular the gluten network of the dough. This process of immobilizing is also referred to as encapsulation or embedding herein.

Preferably, glycerol and/or coconut fat or coconut oil are added to a composition to be extruded. This provides for further enhancement of viability and/or stability of rod-shaped bacteria according to the present invention as comprised in the composition to be extruded during the extrusion process and/or obtained by any downstream processing of the obtained extrudate. Accordingly, in a further aspect, the present invention relates to a process of extruding a composition comprising rod-shaped probiotic bacteria or rod-shaped fermentation bacteria, wherein, prior to the step of extruding, glycerol and/or coconut fat or coconut oil are added to said composition comprising said bacteria. Also provided is the use of glycerol and/or coconut fat or coconut oil for enhancing viability, stability and/or shelf-life of rod-shaped bacteria according to the present invention in an extrudate, glycerol and/or coconut fat or coconut oil being present in the extrudate during the extrusion process.

Spheres can be produced for example by mixing the bacteria in either wet or dry form with a protective binding material, such as for example flours, starches, cellulosic materials or the like, and a sufficient amount of liquid to obtain crumb like particulates that can be compressed into pellets and/or further processed, e.g. in a spheronizer, resulting in particulates having spherical shapes and containing the bacteria of the invention.

Lyophilisates are compositions obtained by freeze-drying. Means and methods for freeze-drying are known in the art and at the skilled person's disposal; see also the enclosed Examples.

The term “frozen preparations” refers to preparations comprising rod-shaped bacteria as defined herein above and stored at temperatures below 0° C., preferably in the range from −10 to −30° C., most preferably about −18 to −20° C.

Preferably, said extrudate is a dough and/or comprises flour and water.

In a further aspect, the present invention provides a method of preparing an extrudate, lyophilisate or frozen preparation, said extrudate, lyophilisate or frozen preparation comprising cells of rod-shaped probiotic bacteria or rod-shaped fermentation bacteria, said method comprising (aa) the method of establishing an average volume and/or average length-to-diameter ratio as defined above or (ab) the method of increasing viability, stability, shelf-life, DNA replication, septum formation and/or division of cells as defined above, and (b) a step of extruding, lyophilising and/or freezing, respectively.

In a further aspect, the present invention provides a composition comprising or consisting of rod-shaped probiotic bacteria and/or rod-shaped fermentation bacteria with an average volume below 3 μm³, preferably between 2 and 3 μm³, more preferably between 1.4 and 2 μm³, and most preferred between 1.1 and 1.4 μm³ and/or an average length-to-diameter ratio below 5, preferably below 4 or below 3 or below 2.5, more preferably below 2.2 or below 2.1 or below 2.0, and most preferred below 1.8.

In a preferred embodiment, said composition is selected from culture medium, beverage, food for human or animal consumption, feed, dietary supplement, biocontrol agent, medicinal product, extrudate, lyophilisate, frozen preparation and dried preparation.

Preferred culture media are those disclosed herein above, in particular MRS and GEM media, wherein said composition according to the present invention, to the extent it relates to culture media, comprises or consists of medium such as MRS or GEM medium and rod-shaped bacteria as defined herein above.

Examples of beverages and foods as well as dietary supplements include yogurt, cheese, curdled milk and products obtained therefrom and probiotics. Further examples are cereal-based products such as ready-to-eat cereals including cornflakes; bars such as chocolate bars; and muesli. Particularly envisaged is the addition of extrudates as disclosed herein to such preparations.

Biocontrol agents as well as preferred embodiments thereof (biopesticides, biopreservatives) are discussed above.

Furthermore provided is a composition comprising or consisting of rod-shaped probiotic bacteria and/or rod-shaped fermentation bacteria, which composition is obtainable or obtained by (i) the method of establishing an average volume and/or average length-to-diameter ratio as defined above; (ii) the method of increasing viability, stability, shelf-life, DNA replication, septum formation and/or division of cells as defined above; or (iii) the method of preparing an extrudate, lyophilisate or frozen preparation as defined above.

In preferred embodiments of methods or compositions of the present invention, preferred rod-shaped probiotic bacteria or rod-shaped fermentation bacteria are as defined further above.

In a further aspect, the present invention provides a method of preparing a cell culture medium, said method comprising treating fat stock peptone, preferably peptone of porcine origin, more preferably a peptic digest of porcine origin in the presence of a reducing sugar such as glucose, fructose, galactose, maltose and lactose at temperatures between 100° C. and 130° C. for at least 15 minutes.

Generally speaking, the higher the temperature, the shorter the required time of treatment at a given temperature. In particular, heat treatment can be performed under standard autoclaving conditions (121° C., 20 min) or by cooking (100° C.) of the medium, wherein the incubation time is preferably more than 1 h, more than 4 h, more than 6 h, or 8 h. An indication for a sufficient heat treatment at temperatures below 120° C. can be the photometric measurement of the absorbance at a wavelength of 420 nm and comparison of the absorbance with standard autoclaving conditions (120° C., 20 min), wherein said absorbance value is preferably above 1, more preferably above 2, and most preferably above 2.9.

The inventor surprisingly found that treating peptone and a reducing sugar together by heating is particularly beneficial in terms of culturing according to the present invention; see also Example 6.

Without wishing to be bound by a specific theory, it is considered that this finding may be correlated directly or indirectly to the generation of beneficial reactants during heat treatment, such as the generation of Maillard reaction products.

The Maillard reaction is classified as non-enzymatic browning, a chemical reaction between an amino acid, peptide or protein and a reducing sugar that condense and progress into a highly complex network of partially unknown reaction products that are collectively known as Maillard reaction products. The Maillard reaction is influenced by many factors such as temperature, time, pH, water activity and reactant source and concentration (e.g. Wijewickreme, A. N. and Kitts, D. D. (1997) Influence of Reaction Conditions on the Oxidative Behavior of Model Maillard Reaction Products. Journal of Agricultural and Food Chemistry, 45, 4571-4576). The antioxidant activity of Maillard reaction products derived from a heated sugar-protein system is well studied (e.g. Jing, H. and Kitts, D. D. (2002) Chemical and biochemical properties of casein-sugar Maillard reaction products. Food Chem Toxicol, 40, 1007-1015; Yeboah, F. K., Alli, I. and Yaylayan, V. A. (1999) Reactivities of D-glucose and D-fructose during glycation of bovine serum albumin. J Agric Food Chem, 47, 3164-3172), and potentially influences the growth behavior.

Maillard reaction products might improve the quality of the culture medium by the generation of a DNA-breaking activity (Hiramoto, K., Kido, K. and Kikugawa, K. (1994) DNA Breaking by Maillard Products of Glucose-Amino Acid Mixtures Formed in an Aqueous System. Journal of Agricultural and Food Chemistry, 42, 689-694). This DNA breaking activity might act on medium components and improve the supply of the bacteria with DNA cleavage products, nucleotides and derivates thereof, which are generated during heating of the medium and/or after heating, during growth in said medium. Rogers et al. (Rogers, D., King, T. E. and Cheldelin, V. H. (1953) Growth stimulation of Lactobacillus gayoni by N-D-glucosylglycine. Proc Soc Exp Biol Med, 82, 140-144) found that the omission of glucose or acid hydrolyzed casein in the growth medium during heat-sterilization reduced the growth stimulation of Lactobacillus gayoni, an effect that is similar to the one found here (see also Example 6). However, in the contribution of Rogers et al. only the growth behavior, recorded as optical density (OD at 600 nm) in the culture broth, was described and no relation to any further cell properties (e.g. cell morphology or stability) is suggested.

### THE FIGURES SHOW

FIG. 1: Cell concentration and cell volume of Lb. acidophilus NCFM grown in media of different manufacturers or compositions for 16 h. Number of independent experiments is indicated in brackets. Determinations in duplicate are stated as mean value± maximum and minimum. For multiple determinations, values are stated as mean value± S.D. The outstanding performance of media comprising Bacto™ Proteose Peptone No. 3 (“GEM Bacto Peptone No. 3” and “MRSD”) is evident. Data are stated in German decimal number format.

FIG. 2: Linearized D-values (D=decimal reduction time) of freeze-dried Lb. acidophilus NCFM preparations as a function of the storage temperature. Culture broth was propagated in GEM containing soy peptone and MRSD. Each value is the mean of at least three storage times at the corresponding temperature.

FIG. 3: Cell volume distribution and phase contrast pictures of Lb. acidophilus NCFM grown for 16 h in GEM containing soy peptone (A), GEM containing Proteose Peptone No. 3 (B) and MRSD (C). The cell concentration (CC) and the mean cell volume (CV) are stated for each culture. Bar dimension: 100 μm. Significantly smaller average cellular volumes (CV) as well as smaller L/D ratios are observed with GEM medium comprising Proteose Peptone No. 3 and MRSD.

FIG. 4: Viability loss of freeze-dried Lb. acidophilus NCFM preparations after storage at 37° C. The mean residual moisture content for all samples after freeze-drying was 3.3%±0.2%. The averaged weight-shift were for samples stored at a relative humidity of 11.3% (A)+0.9±0.3% (w/w) and for samples stored in a dry and gas-tight manner (B)+0.3±0.1% (w/w).

FIG. 5: Bacterial die-off of Lb. acidophilus NCFM during repeated extrusion processes. Determinations were done in triplicate and are stated as mean value±S.D.

FIG. 6: Total cell concentration and mean cell volume of Lb. acidophilus grown for 18 h in different heated MRS(D) media. Additionally, the degree of browning of the applied media is indicated as extinction (or absorbance) at 420 nm. It is illustrated that a heat treatment of even 20 min at 121° C. or 8 h at 100° C. is necessary to reach the full stimulating effect (small cell volumes and high cell concentration). This stimulating effect correlates with the resulting browning of the medium, probably caused by Maillard reaction products.

FIG. 7: Total cell concentration and mean cell volume of Lb. acidophilus grown for 18 h in MRS(D) media, where chosen components were autoclaved separately from the bulk medium and supplemented afterwards. Additionally, the antioxidative capacities and the browning of the applied media are stated. It appears from the data that the bulk medium has to be heat treated in presence of glucose and Peptone No. 3 to reach the full stimulating effect (small cell volumes and high cell concentration. Data are stated in German decimal number format.

The following examples illustrate the invention but should not be construed as being limiting.

### MATERIAL AND METHODS FOR THE FOLLOWING EXAMPLES

### Strains and Media

Lactobacillus acidophilus NCFM was obtained from Danisco A/S in Copenhagen, Denmark. For long-time storage, bacteria were maintained as glycerol-stocks (33% v/v) at −70° C. Prefabricated MRS medium according to de Man et al. (1960) (Difco™, Becton Dickenson GmbH, Heidelberg, Germany), here called MRSD, was used for cultivation. The MRSD contained per liter 10 g Proteose Peptone No. 3, 10 g beef extract, 5 g yeast extract, 20 g dextrose, 1 g Polysorbate 80, 2 g ammonium citrate, 5 g sodium acetate, 0.1 g magnesium sulfate, 0.05 g manganese sulfate and 2 g dipotassium phosphate. For single experiments prefabricated MRS media from other fabricates, but with same concentrations of the ingredients, were used. Those are indicated as MRSR (Carl Roth GmbH & Co. KG, Karlsruhe, Germany), MRSA (Applichem GmbH, Darmstadt, Germany) and MRSS (Scharlau Chemie S. A., Sentmenat, Spain). MRSS was investigated with 0.2% glucose or 0.2% lactose as carbon source.

Further, a general edible medium (GEM), that was developed at the VTT (Technical Research Centre of Finland) [Saarela et al. (2004)] was used. The GEM contained per liter 30 g soy peptone (Serva Elektrophorese GmbH, Heidelberg, Germany), 7 g yeast extract (Serva), 20 g dextrose (Carl Roth), 0.4 g dipotassium phosphate (Carl Roth), 1 g potassium dihydrogen phosphate (Carl Roth), 1 g magnesium sulfate (Merck) and 1 g polysorbate 80 (Carl Roth). In some experiments, the soy peptone in the GEM was replaced by diverse peptones: Proteose Peptone No. 3 (Difco™ or equivalently Bacto™, Becton Dickenson), Soy Peptone (Fluka Chemie GmbH, Oberaching, Germany), Soytone (Difco™, Becton Dickenson), Tryptone (Bacto™, Becton Dickinson), Casitone (Merck KGaA, Darmstadt, Germany). All media were sterilized in 1 liter bottles at 121° C. for 20 minutes as complete composition.

Cultivation media and conditions as defined above have been used for Lactobacillus acidophilus, Lactobacillus casei subsp. rhamnosus, Lactobacillus delbrückii subsp. lactis, Lactobacillus delbrückii subsp. bulgaricus, Lactobacillus johnsonii La1, Lactobacillus rhamnosus GG and Lactobacillus salivarius subsp. salivarius.

### Culture Conditions

For the preparation of precultures, 50 ml of MRSD were inoculated with 2 ml of a glycerol stock culture and incubated for 6 h at 37° C. Main batch-fermentations were inoculated with 1% (v/v) preculture and incubated for 16 h to stationary growth phase in stand cultures unless stated otherwise. All fermentations were done at 37° C.

### Sample Preparation for Freeze Drying

For the preparation of lyophilisates, samples were harvested, separated via centrifugation (8 min, 5000×g) and the supernatant replaced by the same volume of the cryo- and lyoprotective matrix LyoA, containing (w/w) 1.5% gelatine, 1% glycerol, 5% maltodextrin (Glucidex 12®) and 5% lactose monohydrate [Wesenfeld (2005) (Vitalität and Stabilität von probiotischen Mikroorganismen nach der Gefriertrocknung (Lyophilisation); Dissertation an der Fakultät für Prozesswissenschaften der Technischen Universität Berlin)]. These mixtures were aliquoted in 1 ml proportions in 5 ml glass vials, frozen at −70° C. for at least 20 h and lyophilized (Gamma A, Martin Christ Gefriertrocknungsanlagen GmbH, Osterode, Germany) for 28 h to a minimal pressure of 0.022 mbar with the following shelf-temperature-profile: 22 h−20° C., 3 h+10° C., 3 h+30° C.

### Sample Preparation for Encapsulation Experiments

Encapsulation of bacteria was realized by incorporation of a native culture broth in a durum wheat flour matrix. For dough preparation, a 16 h grown culture was cooled in ice-water below 10° C., mixed with durum wheat flour in a ratio of 1 to 3 (g/g) and kneaded manually with a hand-held blender. Thereby the flour was given gradually into the vessel, making sure that homogenous crumby dough was produced. The resulting dough was transferred in the mixing tank of a single screw pasta extruder (PN 100, Haussler GmbH, Heiligkreurthal, Germany) and extruded through 76×0.8 mm Teflon-coated dies with a total die opening area of 38.2 mm² at a constant mass flow of 112.5 g/min. A pasta cutting device was used for pelletization, resulting in pellets of about 4-5 mm in length. All samples were taken at least in triplicate.

### Determination of the Total Cell Count and Cell Volume Distribution

Cell count and cell volume distribution were determined with the Beckman Multisizer™ 3 Coulter Counter® (Beckman Coulter GmbH, Krefeld, Germany). Pulse data were converted to size features by the Multisizer™ 3 software version 3.53. Additionally, cell morphologies were controlled by microscopy (Axioskop 40 FL, Carl Zeiss GmbH, Germany).

### Determination of Cell Survival

Colony forming units (cfu) were determined by the plate count method on MRS-agar (Applichem). Plates were incubated aerobically at 37° C. for 48-72 h. Lyophilized samples were rehydrated in 0.85% NaCl-solution before serial dilution. Pellets with encapsulated bacteria were rehydrated 1:10 (w/w) in prewarmed (37° C.) 0.85% NaCl-solution. Sample tubes were clamped on a tube adaptor and mixed automatically for 30 min at 37° C. at maximum frequency (Vortex-Genie 2, Scientific Industries Inc.). Solution with the dissolved dough was used for decimal dilutions and plated as mentioned above. The viability loss during storage was normally indicated as the logarithm of the cfu per gram after storage (N) divided by the initial number of cfu per gram at the beginning of storage (N₀). Same calculation was applied for samples before (N₀) and after (N) the encapsulation by extrusion process.

### Storage of Bacteria Preparations

Survival rates of bacteria preparations after storage were evaluated using the accelerated shelf-life testing (ASLT) method [Achour et al. (2001) (Application of the accelerated shelf life testing method ASLT to study the survival rates of freeze-dried Lactococcus starter cultures; Journal of Chemical Technology & Biotechnology, 76, 624-628); King et al. (1998) (Accelerated storage testing of freeze-dried and controlled low-temperature vacuum dehydrated Lactobacillus acidophilus; J Gen Appl Microbiol, 44, 160-165)], predicting that the Arrhenius relationship is appropriate for characterization of the shelf-life behavior. Therefore, freeze-dried preparations as well as dough-encapsulated pellets were stored in the dark in dry glass vials closed by gas-tight caps or in an atmosphere with a defined relative humidity. In latter case open vials were stored in a desiccator filled with a saturated lithium chloride (Merck) solution, resulting in a relative humidity of 11.3% [Greenspan (1977) (Humidity fixed points of binary saturated aqueous solutions; J Res Natl Bur Stand A., 81, 89-96)].

### D and Z-Value

To compare the storage behavior of different propagated bacteria, the D_{T} (subscript, upper case T) and the z-values were calculated. D_{T} is the D-value (time required to obtain one Log variation in population) for a given storage temperature T [° C.] after a give n storage time t [h] and indicated in hours. z-value is the temperature span required to obtain a 10-fold variation in D-values indicated in degree Celsius.

### EXAMPLE 1

### Influence of the Growth Medium on the Cell Morphology of Rod-Shape Bacteria

Lb. acidophilus NCFM was grown in different prefabricated MRS media and in GEM for 16 h.

The stated time was chosen to guarantee that the populations reached the stationary growth phase and therefore phenomena as different degrees of chain generation, caused by diverse growth and cell division rates in the exponential growth phase, are blanked out.

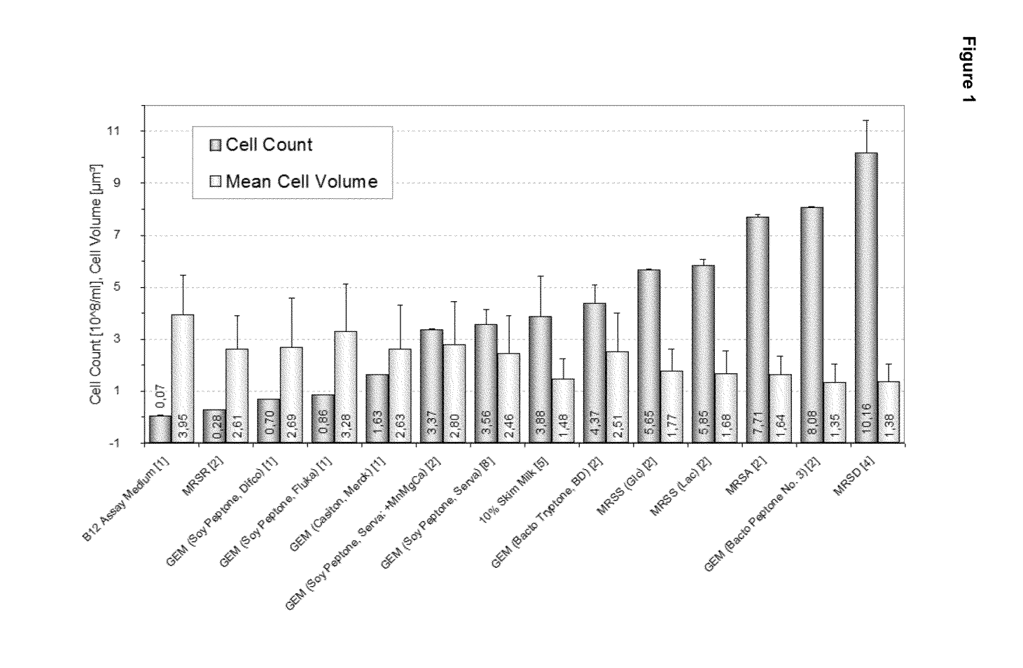

The results are illustrated in FIG. 1, whereby the data are plotted in sequence of increasing cell counts. Particle and cell count analysis revealed that different media lead to significant variations for cell size shape and total cell count of Lb. acidophilus NCFM, reaching from 2.8*10⁷ (MRSR) to 1.0*10⁹ (MRSD) cells per ml with corresponding mean cell volumes of 2.61 to 1.38 μm³, respectively (FIG. 1). In general, mean cell volume increases with decreasing cell count.

To investigate the impact of the peptone on growth behavior and cell morphology, Lb. acidophilus NCFM was propagated in GEM were the standard soy peptone was replaced by five other chosen peptones, including two other soy peptones, two peptones from caseine and the Proteose Peptone No. 3 (see above and FIG. 1). In the modified GEM variation reached from 6.9*10⁸ cells per ml for the tested Soytone from Difco™ to 8.1*10⁸ cells per ml for the Proteose Peptone No. 3. The results demonstrate the high impact of the containing peptone on cell count and cell size. Further, it is obvious that the utilization of media, which include Proteose Peptone No. 3, leads to the highest cell counts as well as smallest mean cell areas.

Other Strains and Species.

Similar observations (outstanding performance of Proteose Peptone No. 3, in particular of MRSD medium) have been observed for Lactobacillus acidophilus, Lactobacillus casei subsp. rhamnosus, Lactobacillus rhamnosus GG and Lactobacillus delbrückii subsp. lactis, thereby demonstrating that Proteose Peptone No. 3 has beneficial effects across a variety of species and strains.

### EXAMPLE 2

### Application of an Accelerated Storage Test for Two Morphologic Diverse Populations

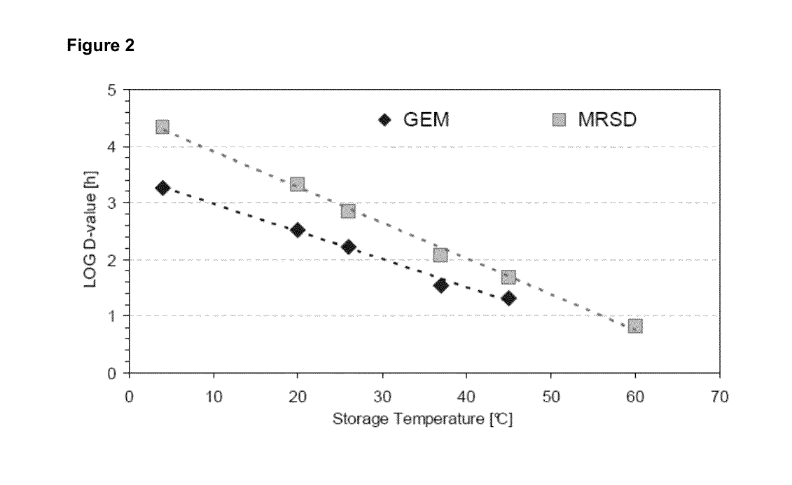

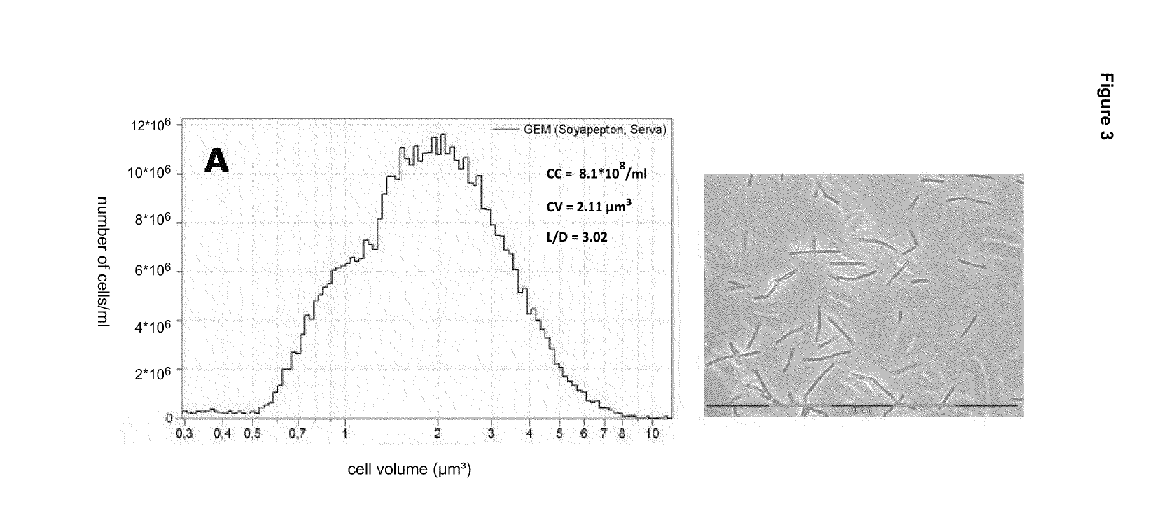

To establish an accelerated shelf life test (ASLT), freeze-dried preparations of Lb. acidophilus NCFM were stored at different temperatures (4, 20, 26, 37, 45 and 60° C.) and analyzed frequently over a time period from 2 days (60° C.) to 520 days (4° C.). These test series were performed for bacteria grown in GEM comprising soy peptone and MRSD. The plotting of the average Log D-values against the corresponding storage temperatures (FIG. 2) led to regression coefficients higher than 0.99 (Table 1). Preparations of cells propagated in MRSD are more stable than those grown in GEM. For example, storage at 4° C. results in a difference in log D_{4° C.}-value of 1.06, which is equal to an elevenfold enhanced shelf-life in preparations made of MRSD-cultures than of GEM-cultures.

TABLE 1
Linear model of the storage behavior of freeze-dried preparations of Lb.
acidophilus NCFM propagated in different media.

| Fermentation | | Regression | z- |
|---|---|---|---|
| Medium | Equation | Coefficient (R²) | Value [° C.] |
| GEM | Log D_{T} = 3.471-0.049T | 0.992 | 15.9 |
| MRSD | Log D_{T} = 4.539-0.063T | 0.997 | 20.4 |

Further, preparations from GEM-cultures had a z-value (reciprocal of the slope of regression equation in FIG. 2) of 15.9° C., which is 4.5° C. lower than of MRSD preparations (Table 2). This difference implies that preparations from MRSD-cultures are storable at a temperature which is 4.5° C. higher than preparations from GEM-cultures which maintain the same shelf-life.

### EXAMPLE 3

### Influence of the Peptone on the Stability Behavior During Freeze Drying and Storage

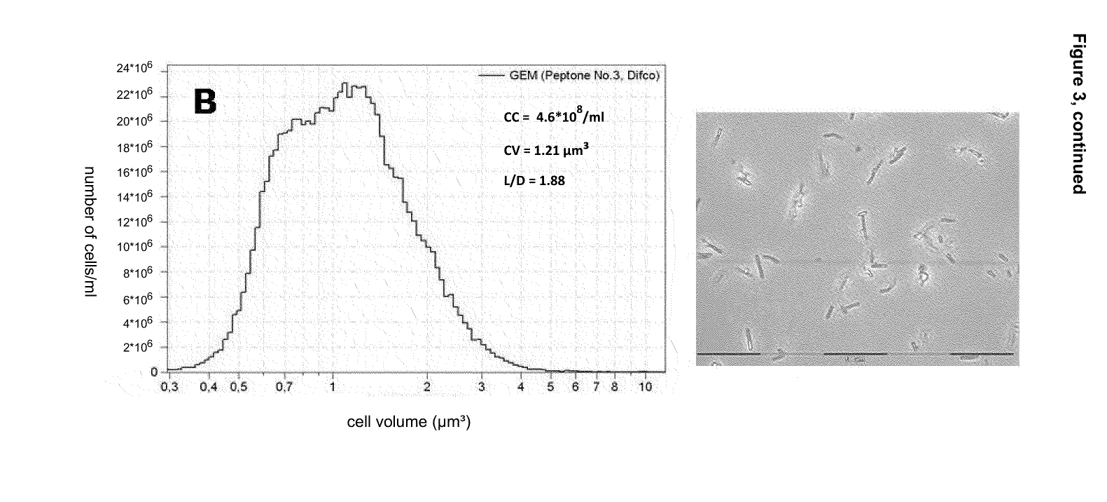

To investigate in particular the influence of the peptone on cell stability after processing, cultures of Lb. acidophilus NCFM were propagated for 16 h in the GEM containing soy peptone (original composition), GEM containing Proteose Peptone No. 3 instead, and MRSD. The characteristics of the cultures are shown in FIG. 3.

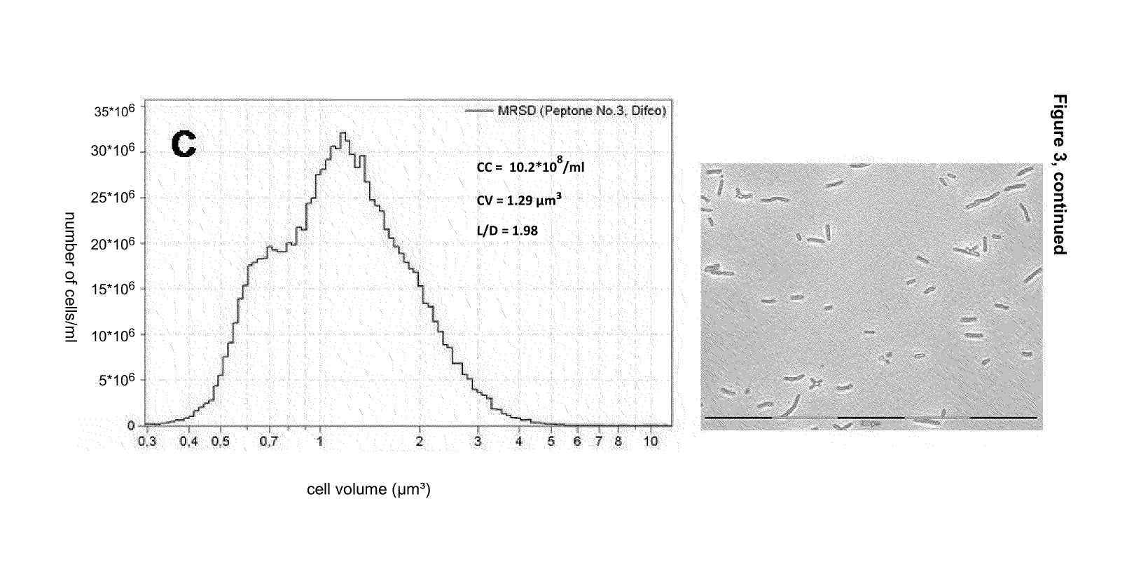

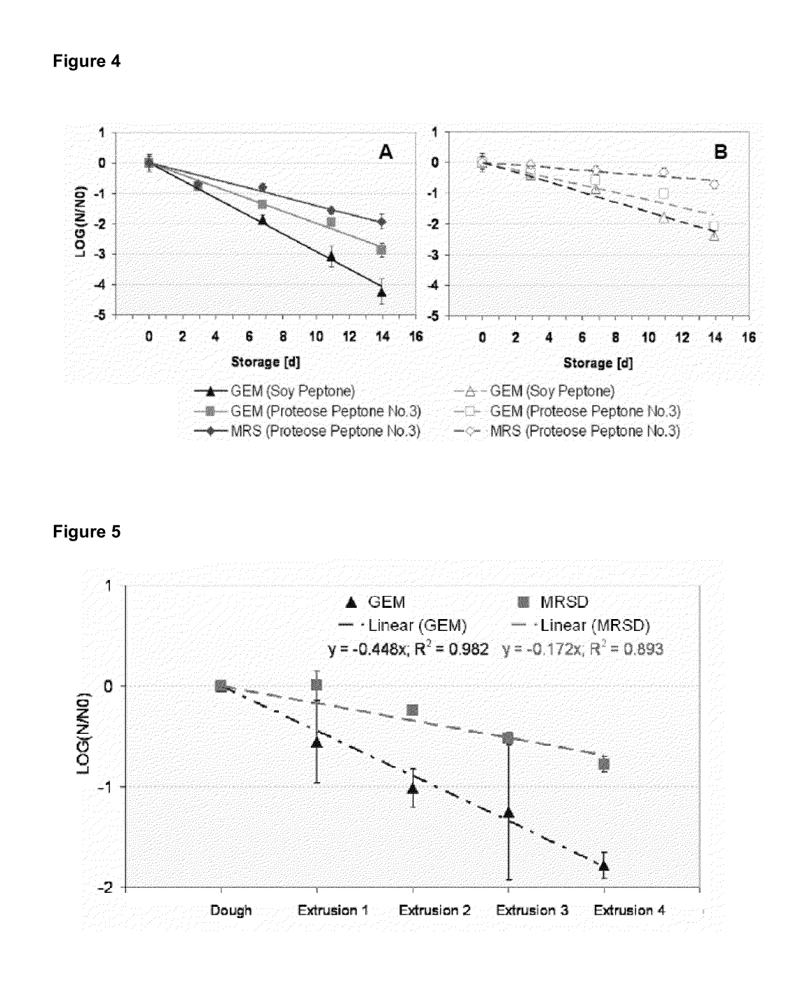

After harvesting, samples were prepared, freeze-dried in 1 ml proportions and stored at different atmospheric conditions at 37° C. (FIG. 4). The cell survivals after freeze drying were for preparations propagated in MRSD, GEM (Proteose Peptone No. 3) and GEM (soy peptone) 104%, 77% and 34%, respectively. The utilization of the Proteose Peptone No. 3 in GEM resulted in a stability enhancement during the freeze-drying procedure as compared to GEM comprising soy peptone instead. This stability tendency was also detectable during the storage of the preparations as seen in FIG. 4 and Table 2. The regression coefficients (R²) for the plotted viability losses in FIG. 4 were between 0.87 and 0.99, indicating a consistent decrease in bacterial viability during storage (Table 2). At both storage conditions the preparation with bacteria grown in GEM with the Proteose Peptone No. 3 instead of the soy peptone were distinctly more stable with an increase of the D_{37° C.}-values of about 40% (85 to 115 h and 168 to 232 h; Table 2). The mean cell sizes of the populations grown in GEM with Peptone No. 3 and MRSD were approximately similar (FIG. 3).

The stabilities of MRSD-cultures were still higher than for cultures grown in GEM with the same peptone. So the replacement of the soy peptone with the Proteose Peptone No. 3 in GEM resulted again in an enhancement of the bacterial stability during storage in dried state, but this stability enhancement, elucidated by the D_{37° C.}-values, did not reach the values of the MRSD-cultures. Especially the preparations stored under conditions of minimal water exchange conditions resulted in the highest averaged D_{37° C.}-value of 1048 h (FIG. 4B, Table 2).

TABLE 2
Results of the accelerated storage test for freeze-dried Lb. acidophilus NCFM
preparations. Bacteria were grown for 16 h in the three stated media and stored
at 37° C. as indicated. RH: Relative humidity, RM: Residual moisture content.

| | | | Slope of | | Averaged RM | Averaged |
|---|---|---|---|---|---|---|
| | | Storage | Mortality Rate | | after Freeze-Drying | D_{37° C.}-Value |
| Medium | | Atmosphere | [LOG(N/Nₒ)/day] | R² | [g_{Water}/g_{Sample}] | [h] |
| GEM_{(SoyPeptone)} | A | RH 11.3% | y = −0.2918x | 0.994 | 3.36% | 85 |
| GEM_{(SoyPeptone)} | B | gas-tight closed | y = −0.1614x | 0.978 | 3.48% | 166 |
| GEM_{(PeptoneNo.3)} | A | RH 11.3% | y = −0.1982x | 0.980 | 3.10% | 115 |
| GEM_{(PeptoneNo.3)} | B | gas-tight closed | y = −0.1227x | 0.878 | 3.37% | 232 |
| MRS_{(PeptoneNo.3)} | A | RH 11.3% | y = −0.1583x | 0.977 | 3.11% | 161 |
| MRS_{(PeptoneNo.3)} | B | gas-tight closed | y = −0.0420x | 0.868 | 3.14% | 1048 |

The averaged weight-shift of samples stored at a relative humidity of 11.3% and in a gas-tight manner with snap caps was +0.9±0.3% (w/w) and +0.3±0.1% (w/w), respectively. It can be estimated that these weight-shifts are caused solely by water sorption of the sample-matrix during vapor equilibration with the surrounding atmosphere. The higher water absorption in the samples stored at a relative humidity of 11.3% resulted in enhanced water activities in the preparations and so to an increase in degradation reactions resp. bacterial die-offs (FIG. 4 A and B, Table 2). The presented results indicate the high influence of the relative humidity resp. water activity in the existing atmosphere for the bacterial viability during storage.

### EXAMPLE 4

### Influence of the Peptone on the Stability Behavior During Freeze Drying with Different Protective Formula

In the course of lyophilization, protection matrices may be employed. One option is the addition of 10% skimmed milk. Another protection matrix designated LyoA has been described in Wesenfeld (2005). The effects of 10% skimmed milk and LyoA in conjunction with either MRSD medium or GEM medium comprising soy peptone have been assessed. For all experiments, the bacteria have been cultivated for 8 hours, centrifuged, and the supernatant replaced with the respective protection matrix. The experimental results are displayed in Table 3 below.

TABLE 3
Influence of the growth medium and the protective matrix on the survival
rate of Lb. acidophilus during freeze-drying. All samples were cultivated
for 8 h, frozen at −70° C. and lyophilized for 24.

| | | Survival | | | |
|---|---|---|---|---|---|
| | | Rate | | | No. of |
| Growth | Protective | after Freeze- | | | independent |
| Medium | Matrix | Drying | ± | SD, MD | Experiment |
| MRSD | 10% Skim Milk | 76.6% | ± | 16.8% | 2 |
| MRSD | LyoA | 88.9% | ± | 8.3% | 3 |
| GEM | LyoA | 58.3% | ± | 15.1% | 3 |
| GEM | 10% Skim Milk | 36.8% | ± | 27.5% | 3 |

The results in Tab.3 illustrate the enhanced stability of Lb. acidophilus when grown in a medium containing the porcine Proteose Peptone No. 3. Additional to the effect of the medium (and therefore the cell population characteristics, see FIGS. 1 and 3), the high influence of the protective matrix is demonstrated.

### EXAMPLE 4

### Behavior of Cells with Different Morphologies During Extrusion Processing

The immobilization of Lb. acidophilus NCFM in a dough matrix was a further processing step for industrial application. The influence of the extrusion process on bacteria with different sizes was investigated. After the batchwise incorporation of the bacteria in the dough, a prearising die-off of 23.4% and 65.0% (referred to the culture broth inclusive dilution caused by flour addition) was detectable for short (grown 16 h in MRSD) and elongated cells (grown 16 h in GEM comprising soy peptone), respectively. To consider the effect of mechanical forces during the extrusion process, the produced pellets were returned into the supply tank of the extruder and extruded again. This procedure was repeated three times. After repeated extrusion processes, for the incorporated MRSD and GEM-broth, the averaged die-off per extrusion step was 33.7% and 62.4%, respectively (FIG. 5). The correlation coefficients of 0.89 (encapsulated GEM culture) and 0.98 (encapsulated MRSD culture) indicate a relative consistent bacterial die-off during each extrusion process.

### EXAMPLE 5

Lb. acidophilus was grown in MRS(D) (MRS comparable to Type Difco: all components are weight out manually; the complex compounds peptone, meat extract and yeast extract are Type Difco) at 37° C. for 18 h.

After solubilization of the MRS(D) components the medium was heat treated at 100° C. for 1, 2, 3, 4, 5, 6, 7, 8 h in closed reaction tubes. As reference MRS(D) medium was autoclaved as specified from medium manufacturer under standard conditions (121° C., 20 min).

All cultivation tubes were weighed empty and with the medium before and after heat treatment for the calculation of weight loss (evaporation). As result, no influencing weight shift was detectable.

The degree of browning in the medium caused by Maillard reaction products were recorded by the absorbance at 420 nm (E420 nm) in a spectral photometer [Morales et al., 2001 (Free radical scavenging capacity of Maillard reaction products as related to colour and fluorescence. Food Chemistry, 72, 119-125.)].

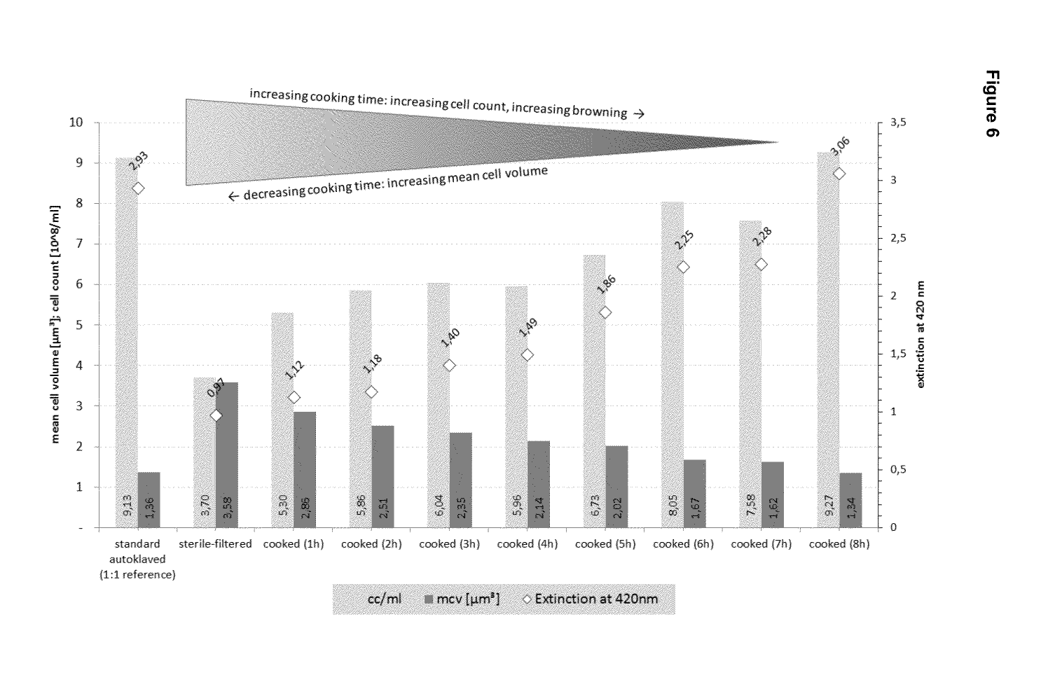

As seen in FIG. 6, there is a linear relation of the cooking time of the MRS(D) medium and the achievable cell concentration as well as with the characteristic mean cell volume. According to these two parameters, a MRS(D) medium that is cooked for 8 h has the same quality as a medium that was autoclaved using standard methods.

### EXAMPLE 6

Lb. acidophilus NCFM was grown in four modified MRS(D) media (MRS comparable to Type Difco: all components are weight out manually; the complex compounds peptone, meat extract and yeast extract are Type Difco). For each medium one component was omitted during steam sterilization. This component was dissolved afterwards in the cooled bulk medium at the original concentration:

Medium 1) Glucose was supplemented after autoclaving of the bulk medium
Medium 2) Peptone No. 3 was supplemented after autoclaving of the bulk medium
Medium 3) Meat extract was supplemented after autoclaving of the bulk medium
Medium 4) Yeast extract was supplemented after autoclaving of the bulk medium

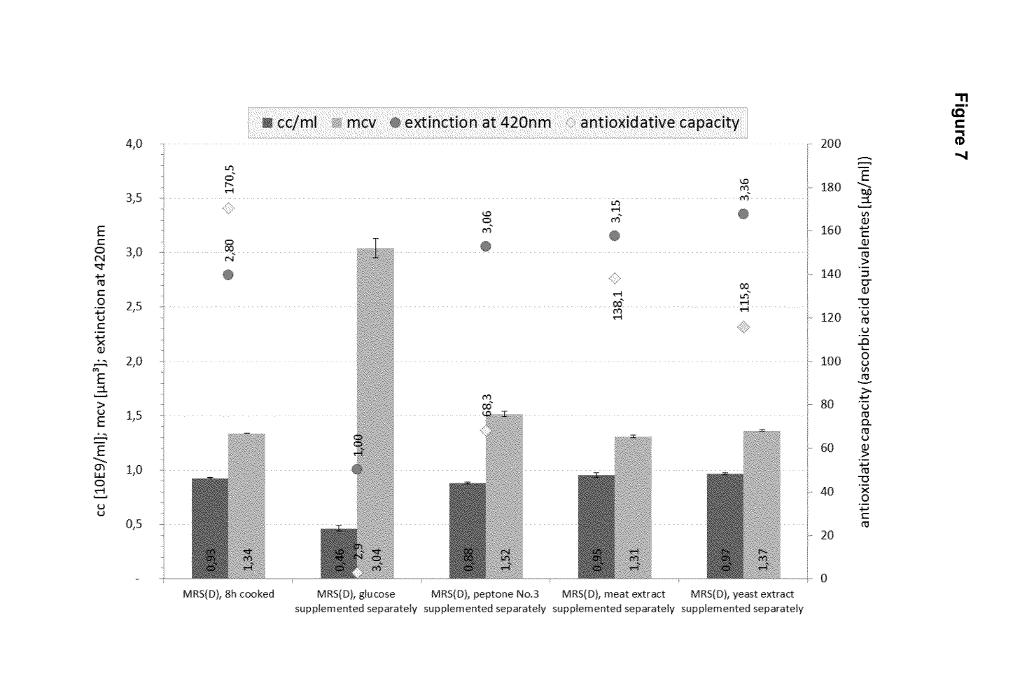

As reference, a MRS(D) medium that was cooked for 8 h are used. Media were characterized additionally by measurement of the antioxidative capacity (PHOTOCHEM® system, Analytik Jena A G, Germany) and measurement of the absorbance at 420 nm. The results of the antioxidative capacity are presented in equivalent concentration units of ascorbic acid for water soluble substances. As seen in FIG. 7, the omission of glucose from MRS(D) during the heat sterilization process has a significant effect on the cell growth. The sterilization without glucose results in media with poor growth and unfavorable cell shapes of Lb. acidophilus. The omission of meat or yeast extract lead to the full stimulating effect of the growth medium equal to the medium where all components together were heat-treated. Without being bound to a specific theory, it is considered that nucleotide derivatives are available to a higher degree in the presence of Maillard reaction products.

1. A method for controlling the volume and/or the length-to-diameter ratio of cells in culture, wherein said cells are cells of rod-shaped probiotic bacteria or rod-shaped fermentation bacteria, the method comprising exposing said cells to a peptone to result in reduced volume and/or length-to-diameter ratio of said cells.

2. The method of claim 1, wherein said peptone is fat stock peptone.

3. The method of claim 1, wherein the average volume of said cells in the presence of said peptone is below 3 μm³ and/or the average length-to-diameter ratio is below 5.

4. The method of claim 1, wherein the viability, stability, shelf-life, DNA replication, septum formation and/or cell division of the cells is increased.

5. A method comprising, culturing rod-shaped Gram positive bacteria in a cell culture medium, determining the average volume and/or the average length-to-diameter ratio of said cells in said culture medium, and selecting the culture medium as suitable for increasing viability, stability, or shelf life of the cells if the average volume of said cells is below 3 μm³ and/or the average length-to-diameter ratio of said cells is below 5.

6. A method of establishing a desired average volume and/or average length-to-diameter ratio of rod-shaped probiotic bacteria cells or rod-shaped fermentation bacteria cells, the desired average volume being below 3 μm³ and the desired average length-to-diameter ratio being below 5, the method comprising culturing said cells in the presence of fat stock peptone such that the cells reach the desired average volume and/or average length-to-diameter ratio.

7. A method of increasing viability, stability, shelf-life, DNA replication, septum formation and/or cell division of cells, wherein said cells are cells of rod-shaped probiotic bacteria or rod-shaped fermentation bacteria, the method comprising culturing said cells in the presence of fat stock peptone such that the volume of the cells reaches an average of less than 3 μm³ and/or the length-to-diameter ratio reaches an average of less than 5.

8. The method of claim 1, wherein said probiotic bacteria or fermentation bacteria are selected from rod-shaped Lactobacillales, rod-shaped Bifidobacteriales, rod-shaped Bacillales and rod-shaped Clostridiales.

9. The method of claim 4, wherein
(a) viability is viability in culture or in a preparation;
(b) stability is stability in a preparation;
(c) shelf-life is shelf-life in a preparation;
(d) DNA replication is DNA replication in culture;
(e) septum formation is septum formation in culture; and
(f) cell division is cell division in culture.

10. The method of claim 9, wherein said preparation is selected from preparations wherein said cells are encapsulated or embedded in a protective matrix, such as extrudates or spheres; lyophilisates; frozen preparations; and dried preparations.

11. A method of preparing an extrudate, lyophilisate or frozen preparation comprising cells of rod-shaped probiotic bacteria or rod-shaped fermentation bacteria, said method comprising culturing said cells in the presence of fat stock peptone such that the cells reach an average volume of less than 3 μm³ and/or average length-to-diameter ratio of less than 5, extruding, lyophilising and/or freezing the cells.

12. A composition comprising rod-shaped probiotic bacteria and/or rod-shaped fermentation bacteria with an average volume below 3 μm³ and/or an average length-to-diameter ratio below 5.

13. The composition of claim 12, wherein said composition is selected from culture medium, beverage, food for human or animal consumption, feed, dietary supplement, biocontrol agent, medicinal product, extrudate, lyophilisate, frozen preparation, and dried preparation.

14. (canceled)

15. A method of preparing a cell culture medium, said method comprising treating fat stock peptone in the presence of a reducing sugar at temperatures between 100° C. and 130° C. for at least 15 minutes.

16. The method of claim 1, wherein the average volume of said cells in the presence of said peptone is between 1.4 μm³ and 3 μm³ and/or the average length-to-diameter ratio is below 1.8.

17. The method of claim 1, wherein the peptone is of porcine origin.

18. The method of claim 6, wherein the peptone is of porcine origin.

19. The composition of claim 12, wherein the average volume of said cells in the presence of said peptone is between 1.4 μm³ and 3 μm³ and/or the average length-to-diameter ratio is below 1.8.

20. The method of claim 15, wherein the fat stock peptone is of porcine origin.

21. The method of claim 15, wherein the reducing sugar is selected from glucose, fructose, galactose, maltose, and lactose.
